(19) United States
(12) Patent Application Publication
(10) Pub. No.: US 2019/0328667 A1
(43) Pub. Date: Oct. 31, 2019

(54) DRUG-POLYMER PARTICLES WITH SUSTAINED RELEASE PROPERTIES

(71) Applicant: University of Washington, Seattle, WA (US)

(72) Inventors: Rodney J.Y. Ho, Seattle, WA (US); Jesse Yu, Seattle, WA (US)

(73) Assignee: University of Washington, Seattle, WA (US)

(21) Appl. No.: 16/348,456

(22) PCT Filed: Nov. 10, 2017
(86) PCT No.: PCT/US17/61157

§ 371 Date: May 8, 2019

Related U.S. Application Data
(60) Provisional application No. 62/420,414, filed on Nov. 10, 2016.

(51) Int. Cl. A61K 9/14; A61K 31/427; A61K 31/513; A61K 31/7068; A61K 9/00

(52) U.S. Cl. CPC A61K 9/146; A61K 31/427; A61K 9/0019; A61K 31/7068; A61K 31/513

(57) ABSTRACT

Drug-polymer complexes, drug-polymer particles, drug-polymer particle formulations, and methods for making and using the complexes, particles, and formulations. The drug-polymer complex is prepared by a melt-quench process in which a combination of a drug and a polymer are heated to or above the melting point of the drug and polymer and then cooled.

### CROSS-REFERENCE TO RELATED APPLICATION

This application claims the benefit of U.S. Application No. 62/420,414, filed Nov. 10, 2016.

### STATEMENT OF GOVERNMENT LICENSE RIGHTS

This invention was made with government support under Grant No. UM1 AI120176 awarded by the National Institutes of Health. The government has certain rights in the invention.

### BACKGROUND OF THE INVENTION

Multi-drug combination therapy has become the standard-of-care for the treatment of diseases, such as caused by infection with Human Immunodeficiency Virus (HIV), and mounting evidence suggests its superiority over mono-drug therapy for the treatment of cancer. Specific to HIV infections, combined antiretroviral therapy (cART) consists of a daily regimen of multiple orally-administered antiretroviral drugs with different viral targets, and its benefits in terms of decreasing drug resistance and increasing therapeutic efficacy are well-established. However, due to challenges with noncompliance and associated viral relapse in patients on contemporary cART, there is urgent need for the development of long-acting anti-HIV drug technologies that can deliver multidrug therapy on a weekly or less frequent basis. Even with the implementation of single oral tablets or capsules containing multiple drugs as standard cART to ease pill burden, each drug in its free form naturally has a different pharmacokinetic profile, creating a challenge in maintaining effective plasma drug concentrations of each drug in concert to optimally suppress HIV without promoting resistance. Additionally, oral combination drugs penetrate poorly into lymph nodes and other lymphoid tissues, resulting in intracellular lymphatic drug concentrations that are low and inconsistent. This drug insufficiency has been linked to residual virus in patients on cART, even if they have low or no detectable virus in blood, which can lead to resurgence of viral levels.

Even with seemingly similar biopolymer or lipid excipients used in drug-nanoparticle formulations, the methods of preparation and the resultant drug-excipients interactions can produce distinct pharmaceutical products with unique pharmacological and toxicological as well as distribution in the target or elimination tissues in the body. Standard procedures for incorporating drugs in enclosed membranes, for example in liposomes, are well-known. However, while liposomes and other enclosed membranes have been proposed as universal carriers for lipid soluble drugs (incorporated into the lipid shell) and water soluble drugs (encapsulated inside the spherical interior), incorporation and encapsulation of multiple drugs has been challenging. Similarly, addition of drugs to biopolymer in buffer or organic mixture do not provide stable association of drug and polymer molecules to produce excipient stabilized structure that exhibit sustained release property in solution.

Even if a drug is successfully associated with lipid or polymeric particles, the resulting particles are often not sufficiently stable for product development. Liposome encapsulation of hydrophilic compounds, which include nucleoside analogue reverse transcriptase inhibitors (RTIs) such as tenofovir (TFV), lamivudine (3TC), and emtricitabine (FTC), which are key components of first-line cART, has proven particularly difficult. Encapsulation of small hydrophilic molecules in traditional liposomes with neutral charge is generally very low, often less than a few percentage points, due to the large amount of external aqueous space relative to the entrapped internal aqueous compartment of small unilamellar vesicles. Attempts to increase the capture of TFV require modification of the membrane content with a positively charged fatty acid. Not only are fatty acids readily removed from liposome membranes by proteins in serum, thus rendering the liposome carrier unstable and ineffective, but the positively charged cationic particles also interact with erythrocytes and other cells in vivo, leading to particle instability and cellular toxicity. Indeed, issues of toxicity associated with positively charged lipids have been a major barrier to clinical application of cationic non-viral vectors 17.

Even for hydrophobic HIV drugs, which should more readily incorporate into lipid membranes, optimization studies with different lipid compositions yield incomplete and uneven degrees of two HIV drugs incorporated in liposomes. In addition, these liposomes, which even include polyethylene glycol modification to improve stability, readily release the drugs when incubated in only 10% serum, with approximately 80% of drug released within the first hour of incubation. The rapid destabilization of liposome-bound drug renders these particles ineffective for transit of drug from the injection site to target tissues. While solid polymeric particles can incorporate multiple hydrophobic drugs with as high as 81% efficiency, these particles are even larger than liposomes and lack an aqueous compartment, limiting their utility in accommodating multi-drug combinations. Additionally, these large polymeric drug carriers and smaller quantum dots often become trapped at the local site of injection and released slowly rather than transiting as a single drug-particle unit to the lymphoid tissues and cells.

A number of variation of and alternatives to standard liposome assembly procedures have been reported. For example, in reverse evaporation vesicle (REV), multilamellar vesicle, unilamellar vesicle, ethanol injection methods of liposome preparation are used. These preparations could provide high degree of incorporation of a single drug molecule, particularly for lipid soluble drugs. However, the ability to capture the water soluble hydrophilic drug in solution is variable and depends on the volume trapped and charge of drug molecule within liposomal enclosed membrane environment.

Intra-liposomal precipitation of doxorubicin-sulfate provides a method for high efficiency drug loading in the liposomes based on the ability of liposomal membrane to entrap charged molecules, such as (NH₄)₂SO₄, and membrane permeability of doxorubicin-HCl. However, this process, referred to as remote loading, is only suitable of limited number of drugs that are permeable and with counter ions that exhibit low solubility. Unfortunately, not all the drugs are suitable for remote loading into liposomes.

To increase encapsulation efficiency of liposomes and lipid-drug nanoparticles in pharmaceutical scale, others have used microencapsulation vehicle methods with either a single or double emulsion approaches with limited success. For example, a microemulsion approach produces varying degrees of reproducible drug levels for incorporation and requires removal of residual organic solvent from water at the final step, which could be difficult and could pose toxicity risk if removal process is incomplete. With single agent incorporation, the drug of interest must be added in an organic (oil) phase (for hydrophobic drug) or a water phase (for water soluble hydrophilic drug). This procedure is difficult to incorporate multiple drugs, particularly those that exhibit different physical characteristics—hydrophobic drug and hydrophilic drugs. A variation of the microemulsion approach is the double emulsion method, whereby a drug and a precipitant such as KCl are placed in two separate water in oil (o/w/o) emulsions with lipidic excipient and a surfactant. Upon mixing of the two o/w/o emulsions, the drug-KCl particles are formed as nano-drug precipitates. These nano-precipitates with a monolayer of lipidic coats are subjected to a second step of coating in organic or w/o emulsion of lipids such as cholesterol and DOTAP. This produces very low efficiency of nanoparticle drug incorporation. For example, for tenofovir (also referred to as PMPA), the final % drug association is less than 3%. Moreover, this approach is designed for a signal drug nanoparticle formation. While the drug loading, based on lipid to drug ratio may be high, the percent of drug wastage, based on the fraction of drug associated from start-to-finish is low.

To improve incorporation of water-soluble drugs that carry a charge, positively charged lipids, such as 1,2-dipalmitoyl-3-trimethylammonium-propane (DPTAP), have been included in the lipid membrane for electrostatic interaction. Unfortunately, positive charge can pose toxicity risk in animals and resulting particle sizes (larger than 100 nm diameter) are more susceptible to rapid clearance and elimination from the body. Similarly, incorporation of a fatty acid, stearylamine (SA), that carries a positive charge in the phosphatidylcholine lipid can increase the association of hydrophilic drugs such as TFV to 70% with large particles up to about 2000 nm in diameter. However, the positively charged particles have positive zeta potentials of 53-93 mV and exhibit mitochondrial toxicity by the same order. Therefore, compositions incorporating charged lipids to facilitate the incorporation of hydrophilic drugs are unlikely to be suitable for clinical development.

Despite the advances in the art, there remains a need for methods to efficiently and inexpensively incorporate multiple, structurally distinct drugs into a single drug delivery vehicle that provides for stable delivery to the intended tissues and targets within the body. In the context of HIV treatment, there remains a need for a drug delivery vehicle that is capable of sustaining drug concentrations in plasma. The present disclosure seeks to address these needs based on strong drug-biopolymer interactions to produce a drug-combination particle and provides further related advantages.

### SUMMARY OF THE INVENTION

The present invention provides drug-polymer complexes, drug-polymer particles, drug-polymer particle formulations, and methods for making and using the complexes, particles, and formulations.

In one aspect, the invention provides a drug-polymer complex.

In one embodiment, the drug-polymer complex comprises:

(a) one or more therapeutic agents; and

(b) a triblock copolymer having a first block that is a hydrophilic block, a second block that is a hydrophobic block, and a third block that is a hydrophilic block, wherein the second block is intermediate the first and third blocks,

wherein the complex is substantially free of water.

As used herein, the term “substantially free of water” refers to a drug-polymer complex that includes less than about 1% by weight water, less than about 0.5% by weight water, or less than about 0.1 percent by weight water. In certain embodiments, the drug-polymer complex that includes less than about 0.1 percent by weight water.

In one embodiment of the above embodiment, the one or more therapeutic agents are substantially amorphous.

In another embodiment, the drug-polymer complex comprises:

(a) one or more therapeutic agents; and

(b) a triblock copolymer having a first block that is a hydrophilic block, a second block that is a hydrophobic block, and a third block that is a hydrophilic block, wherein the second block is intermediate the first and third blocks, wherein the therapeutic agent is substantially amorphous.

As used herein, the term “substantially amorphous” refers to the nature of the therapeutic agent in the complex; greater than about 90% amorphous, greater than about 95% amorphous, or greater than about 99% amorphous. In certain embodiments, the therapeutic agent in the complex is substantially amorphous and has no detectable crystallinity.

In certain embodiments, the one or more therapeutic agents are hydrophobic drugs (e.g., having a Log D from about 3 to about 5).

In other embodiments, the one or more therapeutic agents are hydrophilic drugs (e.g., having a Log D from about −2 to about 1.

In further embodiments, the complex comprises one or more hydrophobic therapeutic agents and one or more hydrophilic therapeutic agents.

In certain embodiments, the one or more therapeutic agents is an antiviral agent. In certain embodiments, the one or more therapeutic agents is an anti-retroviral agent. Representative therapeutic agents include lopinavir, ritonavir, lamivudine, and combinations thereof. In one embodiment, the one or more therapeutic agents are lopinavir, ritonavir, and lamivudine.

In certain embodiments, the triblock copolymer has a first block that is a polyoxyethylene block, a second block that is a polyoxypropylene block, and a third block that is a polyoxyethylene block.

In certain embodiments, the triblock copolymer has the formula:

H(OCH₂CH₂)ₓ(OCH₂CH(CH₃))_{y}(OCH₂CH₂)_{z}OH

wherein x is an integer from about 10 to about 200, y is an integer from 20 to about 80, and
z is an integer from about 10 to about 200. In certain of these embodiments, x is an integer from about 10 to about 150, y is an integer from 20 to about 60, and z is an integer from about 10 to about 150. In other of these embodiments, x is an integer from about 90 to about 120, y is an integer from 40 to about 70, and z is an integer from about 90 to about 120. In one embodiment, x is about 101, y is about 56, and z is about 101.

In certain embodiments, the ratio of therapeutic agent to triblock copolymer is about 10:90 weight:weight. In other embodiments, the ratio of therapeutic agent to triblock copolymer is about 25:75 weight:weight. In further embodiments, the ratio of therapeutic agent to triblock copolymer is about 50:50 weight:weight.

In another aspect of the invention, a drug-polymer particle is provided. The drug-polymer particle comprises the drug-polymer complex as described herein.

In certain embodiments, the particle is a microparticle. In certain of these embodiments, the particle has a particle size from about 1 μm to about 10 μm.

In other embodiments, the particle is a nanoparticle. In certain of these embodiments, the particle has a particle size from about 50 nm to about 300 nm.

In a further aspect, the invention provides drug-polymer particle formulations. In certain embodiments, the formulation is a pharmaceutical composition, comprising the drug-polymer particle described herein and a pharmaceutically acceptable carrier. Suitable carriers include carriers that are suitable for injection, such as saline or dextrose solution.

In other aspects of the invention methods for using the complexes, particles, and formulations are provided.

In one aspect, the invention provides methods for administering a therapeutic agent to a subject. In certain embodiments, the method comprises administering a therapeutically effective amount of the drug-polymer complex as described herein to a subject in need thereof. In other embodiments, the method comprises administering a therapeutically effective amount of the drug-polymer particle described herein to a subject in need thereof.

In another aspect, the invention provides methods for treating a disease of condition. In certain embodiments, the method comprises administering a therapeutically effective amount of a drug-polymer complex as described herein to a subject in need thereof, wherein the disease or condition is treatable by administering the therapeutic agent of the drug-polymer complex. In other embodiments, the method comprises administering a therapeutically effective amount of a drug-polymer particle as described herein to a subject in need thereof, wherein the disease or condition is treatable by administering the therapeutic agent of the drug-polymer particle. In certain embodiments, the one or more therapeutic agents is an antiviral agent. In certain embodiments, the one or more therapeutic agents is an anti-retroviral agent. Representative therapeutic agents include lopinavir, ritonavir, lamivudine, and combinations thereof. In one embodiment, the one or more therapeutic agents are lopinavir, ritonavir, and lamivudine.

In certain embodiments of the above methods, the drug-polymer complex or drug-polymer particle is administered subcutaneously.

In a further aspect of the invention, methods for making a drug-polymer complex are provided. In certain embodiments, the method comprises:

(a) heating one or more therapeutic agents and a triblock copolymer having a first block that is a hydrophilic block, a second block that is a hydrophobic block, and a third block that is a hydrophilic block, wherein the second block is intermediate the first and third blocks to above the melting temperature of the therapeutic agent and triblock copolymer to provide a molten material; and

(b) cooling the molten material to provide a solid drug-polymer complex.

In certain embodiments, heating the one or more therapeutic agents and the triblock copolymer comprises heating to 125° C. at a rate of 5° C./min.

In certain embodiments, cooling the molten material comprises cooling to 4° C.

In certain embodiments, the solid drug-polymer complex is the drug-polymer complex described herein.

In another aspect, the invention provides methods for making a drug-polymer particle. In certain embodiments, the method comprises:

(a) heating one or more therapeutic agents and a triblock copolymer having a first block that is a hydrophilic block, a second block that is a hydrophobic block, and a third block that is a hydrophilic block, wherein the second block is intermediate the first and third blocks to above the melting temperature of the therapeutic agent and triblock copolymer to provide a molten material;

(b) cooling the molten material to provide a solid drug-polymer complex; and

(c) triturating the solid drug-polymer complex in an aqueous medium to provide a suspension of drug-polymer particles in the aqueous medium.

In certain embodiments, the method further comprises subjecting the drug-polymer particles to size reduction to provide drug-polymer particles having a pre-determined size.

In certain embodiments, heating the one or more therapeutic agents and the triblock copolymer comprises heating to 125° C. at a rate of 5° C./min.

In certain embodiments, cooling the molten material comprises cooling to 4° C.

In certain embodiments, the solid drug-polymer complex is the drug-polymer complex described herein.

In certain embodiments, the aqueous medium is deionized water.

In certain embodiments, the drug-polymer particle is the particle described herein.

In certain embodiments, particle size reduction comprises mechanical grinding, sonication, homogenization, microfluidization, and combinations thereof. In certain embodiments, the drug-polymer particle having a pre-determined size is a particle as described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a graphical representation of a triblock copolymer having polyoxyethylene-polyoxypropylene-polyoxyethylene blocks (e.g., poloxamer) and a physical mixture of the copolymer with multiple drugs (i.e., a hydrophilic drug lamivudine and hydrophobic drugs lopinavir and ritonavir) and water. The hydrophilic drug lamivudine and water associate with the hydrophilic portion of the triblock copolymer (polyoxyethylene) and the hydrophobic drugs lopinavir and ritonavir associate with the hydrophobic portion of the triblock copolymer (polyoxypropylene). Fusion of triblock copolymer and hydrophobic and hydrophilic drugs provides a representative multi-drug triblock copolymer particle having strong non-bonding interactions that when hydrated result in colloidal particle formation, improved suspension characteristics, and sustained drug release from the particle. Hydration of the physical mixture reflects only a loose association between the drug and copolymer, and no sustained drug release.

FIGS. 2A and 2B are high resolution microscopic images of an aqueous suspension containing representative drug triblock copolymer particles of the invention (lopinavir and poloxamer F127 particles) formed in accordance with a melt-quench method of the invention. Colloidal and micron-sized particles and crystalline drug are observed having varying morphologies.

FIGS. 3A and 3B are high resolution microscopic images of an aqueous suspension containing drug triblock copolymer particles (lopinavir and poloxamer F127 particles) formed as a physical mixture. Large aggregate particles can be observed with irregular, faceted morphology. Under optimized conditions, the majority of particles (>75%) are observed to be submicron population.

FIGS. 4A and 4B graphically illustrate drug release over time for a representative multi-drug triblock copolymer particle of the invention: melt-quench formed lopinavir (LPV), ritonavir (RTV), lamivudine (3TC), poloxamer F127. Release of lopinavir and ritonavir from the particle is shown in FIG. 4A and release of lamivudine from the particle is shown in FIG. 4B. Drug release was measured under dialysis conditions (37° C.) in water. The results show that the release of each drug was sustained.

FIG. 5 is the powder X-ray diffraction pattern of a representative multi-drug triblock copolymer particle of the invention: melt-quench formed lopinavir (LPV), ritonavir (RTV), lamivudine (3TC), poloxamer F127. Absence of most diffraction peaks indicates a degree of phase transition from a crystalline to an amorphous phase of the individual drugs.

FIG. 6 is the powder X-ray diffraction pattern of physically mixed particles of lopinavir, ritonavir, lamivudine, and poloxamer F127. Individual diffraction peaks as denoted with arrows and indicate significant crystallinity in the particle.

FIG. 7 is a high resolution microscopic image of an aqueous buffer containing representative multi-drug triblock copolymer particles of the invention: melt-quench formed lopinavir (LPV), ritonavir (RTV), lamivudine (3TC), poloxamer F127 particles followed by sonication.

FIG. 8 illustrates the effect of drug:copolymer ratio on lopinavir concentration in suspension for a representative drug triblock copolymer particle of the invention (lopinavir and poloxamer F127 particles) formed in accordance with a melt-quench method of the invention. Lopinavir with varying weight by weight ratio of F127 were prepared by the melt-quench. Drug in suspension was measured in μg/ml. Panel A represents reported lopinavir solubility and measured lopinavir concentrations alone after melt/quench. Panel B represents effect of varied drug:polymer ratios after melt/quench on drug concentration.

FIG. 9 illustrates the effect of drug:copolymer ratio on lopinavir retention in complex for a representative multi-drug triblock copolymer particle of the invention: melt-quench formed lopinavir (LPV), ritonavir (RTV), lamivudine (3TC), poloxamer F127 particles followed by centrifugation. Drug polymer complexes were prepared in accordance with a representative melt-quench method of the invention followed by suspension and separation through centrifugation into coarse (>1 micron) and colloidal (<1 micron) particles. Degree of drug retention in the complexes was evaluated after three hours of dialysis. Data is expressed in percent of lopinavir remaining from dialysis of the supernatant (colloidal), resuspended pellet (coarse), and total complex.

FIG. 10 compares particle size distributions of representative multi-drug copolymer colloidal particles: melt-quench formed lopinavir (LPV), ritonavir (RTV), lamivudine (3TC), poloxamer F127 particles. Supernatant particle size distribution obtained by intensity, volume, and number weighing schemes. Chi squared value for Gaussian distribution=0.471. Total drug:polymer ratio was 25:75.

### DETAILED DESCRIPTION OF THE INVENTION

In certain aspects, the present invention provides a drug-polymer complex, a drug-polymer particle, and a drug-polymer particle formulation. The drug-polymer complex is prepared by a melt-quench process in which a combination of a drug and a polymer are heated to or above the melting point of the drug and polymer and then cooled. The drug-polymer particle is prepared from the drug-polymer complex. The drug-polymer particle formulation includes drug-polymer particles and a suitable carrier. In other aspects, the invention provides methods for making drug-polymer complexes, drug-polymer particles, and drug-polymer particle formulations. Methods for using the drug-polymer complexes, drug-particle particles, and drug-polymer particle formulations are also provided.

The polymer of the drug-polymer complex is a triblock copolymer has a first block that is a polyoxyethylene block, a second block that is a polyoxypropylene block, and a third block that is a polyoxyethylene block. As used herein, the terms “polymer” and “copolymer” are used interchangeably and refer to a triblock copolymer having a first block that is a hydrophilic block, a second block that is a hydrophobic block, and a third block that is a hydrophilic block, wherein the second block is intermediate the first and third blocks (e.g., a triblock copolymer having a first block that is a polyoxyethylene block, a second block that is a polyoxypropylene block, and a third block that is a polyoxyethylene block).

Drug-Polymer Complexes

In one aspect, the invention provides drug-polymer complexes. As used herein, the term “drug-polymer complex” refers to a combination of a drug and a polymer that is formed by heating the combination of the drug and polymer to above the melting point of the polymer (fusion), which has the effect of removing associated water from the drug and/or polymer to prove a melt (molten combination) that is then cooled (quenched) to provide the complex. The process for forming the drug-polymer process is referred to as a “melt-quench” process and provides a melt-quench product (i.e., drug-polymer complex). The melt-quench product may be characterized by the amorphous nature of the drug component of the complex. In the complex, the drug has an increased amorphous character relative to other similarly constituted complexes that are not formed by the melt-quench process described herein (e.g. physical mixtures). The complex also has a decreased amount of associated water relative to other similarly constituted complexes that are not formed by the melt-quench process. The complex also has the property of sustained drug release when the complexes are further formulated as particles in, for example, an aqueous suspension.

The drug-polymer complexes of the invention may include more than a single drug (i.e. two, three, or more different therapeutic agents). By virtue of the polymer component, the drug-polymer complexes may include both one or more hydrophobc drugs (i.e., having a Log D greater than about 3 (e.g., from about 3 to about 5) and one or more hydrophilic drugs (i.e., therapeutic agents having a Log D less than about 1 (e.g., from about −2 to about 1).

Partition-coefficient (P) or distribution-coefficient (D) is the ratio of concentrations of a compound in a mixture of two immiscible phases at equilibrium (i.e., octanol and water). For pharmaceutical application, the Log P or Log D measured at the physiological pH 7-7.4 is an important consideration. This ratio is a measure of the difference in solubility of the compound in these two phases. The partition-coefficient refers to the concentration ratio of un-ionized species of compound whereas the distribution-coefficient refers to the concentration ratio of all species of the compound (ionized plus un-ionized). The partition coefficient Log P is a constant for the molecule under its neutral form. The distribution coefficient Log D takes into account all neutral and charged forms of the molecule. Because the charged forms hardly enter the octanol phase, this distribution varies with pH.

As used herein, drug-polymer complexes that include two or more (e.g., two or three) therapeutic agents are referred to as “multi-drug-polymer complexes.” These drug-polymer complexes advantageously deliver their component multiple drugs in a sustained release fashion.

The nature of the drug (therapeutic agent) useful in the complexes of the invention is not particularly critical. Suitable drugs include those that can be melt-quenched processed with the polymers of the complexes. In certain embodiments, the drugs are antiviral drugs and antiretroviral drugs, such as HIV drugs. Representative drugs useful in the complexes of the invention include lopinavir, ritonavir, lamivudine, and combinations of these drugs in the multi-drug polymer complexes. In other embodiments, these drugs can be chemotherapeutic in nature and include compounds suitable for melt-quench processing such as dourinavir, atazanvir, dolutigravir, raltigravir, efevirenz and azidothymidine, lamivudine, emtricitabine tenofovir.

The polymer component of the drug-polymer complexes of the invention is a copolymer. The polymer component is a triblock copolymer having a first block that is a hydrophilic block, a second block that is a hydrophobic block, and a third block that is a hydrophilic block, wherein the second block is intermediate the first and third blocks. More specifically, the triblock copolymer has a first block that is a polyoxyethylene block, a second block that is a polyoxypropylene block, and a third block that is a polyoxyethylene block. The triblock copolymer has the following formula:

H(OCH₂CH₂)ₓ(OCH₂CH(CH₃))_{y}(OCH₂CH₂)_{z}OH

where x is an integer from about 10 to about 200, y is an integer from 20 to about 80, and z is an integer from about 10 to about 200. In certain embodiments, x is an integer from about 10 to about 150, y is an integer from 20 to about 60, and z is an integer from about 10 to about 150. In other embodiments, x is an integer from about 90 to about 120, y is an integer from 40 to about 70, and z is an integer from about 90 to about 120. In certain embodiments, x and z are about the same.

Triblock copoloymers of this formula where x and z are the same are commercially available from a variety of sources under the designations Poloxamer or Pluronic. The following table illustrates representative triblock copolymers useful in the drug-polymer complexes of the invention.

| Pluronic | Poloxamer | X | Y | Z |
|---|---|---|---|---|
| L44NF | 124 | 12 | 20 | 12 |
| F68NF | 188 | 80 | 27 | 80 |
| F87NF | 237 | 64 | 37 | 64 |
| F108NF | 338 | 141 | 44 | 141 |
| F127NF | 407 | 101 | 56 | 101 |

The weight average molecular weight ranges of Poloxamers 124, 188, 237, 338, and 407 are 2090 to 2360, 7680 to 9510, 6840 to 8830, 12700 to 17400, and 9840 to 14600, respectively. The weight average molecular weights of Poloxamers 124, 188, 237, 338, and 407 are 2200, 8400, 7700, 14600, and 12600, respectively.

A representative triblock copolymer useful in the drug-polymer complexes of the invention is Poloxamer 407, a triblock copolymer consisting of a central hydrophobic block of polyoxypropylene flanked by two hydrophilic blocks of polyoxyethylene. The approximate lengths of the two polyoxyethylene blocks is 101 repeat units while the approximate length of the polyoxypropylene block is 56 repeat units. This particular compound is also known by the BASF trade name Pluronic F127 or by the Croda trade name Synperonic PE/F 127. As used herein, Poloxamer 407, Pluronic F127, and Synperonic PE/F 127 are referred to as “F127” or “poloxamer F127.”

The ratio of drug to polymer drug-polymer complexes can be varied to vary the characteristics of the complex including drug release over time. In certain embodiments, the drug:polymer ratio is 10:90 weight/weight. In others embodiments, the drug:polymer ratio is 25:75 weight/weight. In further embodiments, the drug:polymer ratio is 50:50 weight/weight. In certain embodiments, 25:75 is preferred due to greatest association efficiency. In one representative example, final ratios by weight are 76:12:3:9 poloxamer:lopinavir:ritonavir:lamivudine.

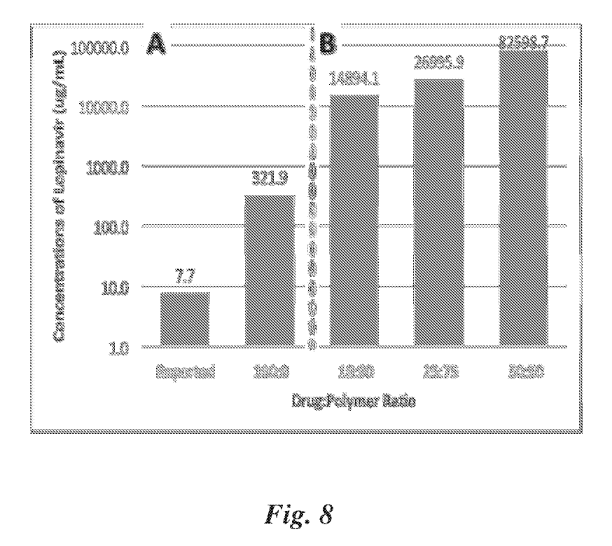

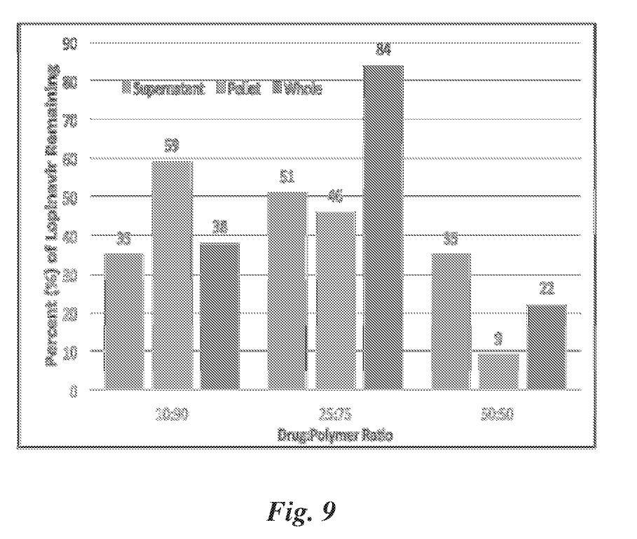

FIGS. 8 and 9 illustrate the effect of drug:polymer ratio on drug release for representative drug-polymer particles of the invention.

Drug-Polymer Particles

In another aspect, the invention provides drug-polymer particles. The drug-polymer particles are prepared from the drug-polymer complexes. The drug-polymer particles can be formulated for administration to advantageously provide sustained drug release.

The drug-polymer particles are prepared by combining drug and polymer at the desired weight:weight ratio (e.g., 10:90, 25:75, 50:50) and heated until fusion. Heating to fusion is effective to remove associated water from the combination (i.e., drug and polymer) and to transform at least a portion of the drug from its crystalline state to an amorphous state. The molten state combination is then cooled to provide a solid (e.g., solid pellet). The solid is then triturated in an aqueous medium (e.g., deionized water) to provide a particle suspension. The particle suspension can be further subjected to particle size reduction to provide drug-polymer particles having the desired size distribution.

In certain embodiments of the melt-quench process, the drug and polymer were mixed at room temperature and heated at a rate of 5° C./min to above the melting temperature of both components (e.g., 125° C.). The molten material was held isothermally for 5 minutes while mixing. The molten material was quenched by removing the heating vessel from the heat source and immediately placing the vessel in ice water (4° C.) to provide the drug-polymer complex product.

Trituration of the drug-polymer complex product in aqueous media provided particles, which can be subject to size reduction. Particle size reduction can be achieved by a variety of size reduction processes. Suitable particle size reduction processes include mechanical processes, such as grinding (e.g. mortar and pestle), sonication (e.g., temperature controlled), homogenization, and microfluidization. Because size reduction processes can transfer heat to the complex that may adversely affect the stable interactions in the complex, care is taken to reduce or avoid heat input to the complex.

Larger particles in buffered suspension can be further sonicated to reduce particle size. Alternatively, sonication can be replaced by other mechanical processes such as homogenization or microfluidization.

The data presented herein in was generated with materials that have gone through size reduction using a mortar and pestle followed by sonication in a temperature controlled water bath for 15 minutes.

In certain embodiments, the invention provides drug-polymer particles have a particle size from about 50 nm to about 300 nm.

In other embodiments, the invention provides drug-polymer particles have a particle size from about 1 μm to about 10 μm.

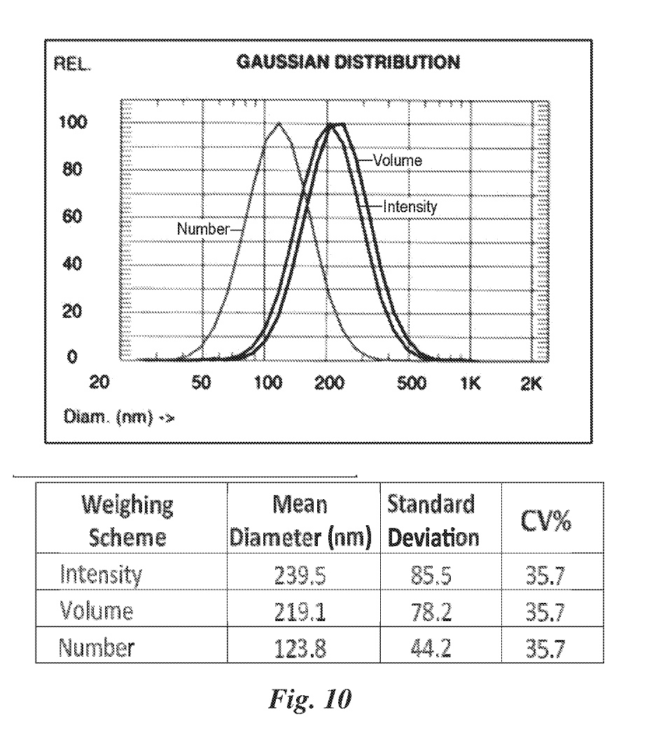

FIG. 10 illustrates particle size and particle size distribution for representative drug-particle particles of the invention.

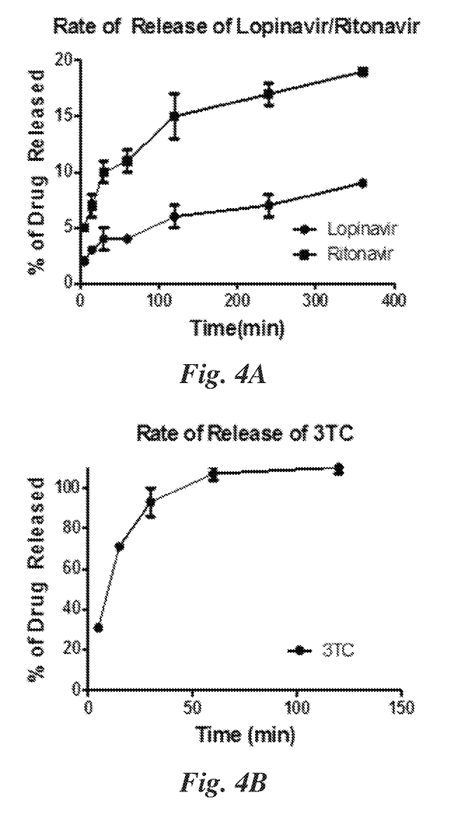

The drug-polymer particles advantageously demonstrate sustained release as shown in FIGS. 4A and 4B.

Drug-Polymer Particle Formulations

In a further aspect, the invention provides drug-polymer particle formulations. The drug-polymer particle formulations include drug-polymer particles and a suitable carrier or diluent. For pharmaceutical compositions, the carrier or diluent is a pharmaceutically acceptable carrier or diluent. Representative pharmaceutically carriers or diluents include carriers or diluents for injection, for example, saline or dextrose solutions.

The amount of particles in the formulation will vary depending on the drug to be delivered and mode of delivery.

Formulations of the invention can be prepared as described above for the preparation of drug-polymer particles using the appropriate carrier or diluent and sizing the particles to the desired particle size and particle size distribution.

Methods of Use

In another aspect, the invention provides methods for using the drug-polymer complexes, drug-polymer particles, and drug-polymer particle formulations. The uses of these compositions derive from the drug component of the complexes, particles, and formulations, as well as the advantageous sustained release of drug from the drug-polymer complex and drug-polymer particles.

As a delivery vehicle, the drug-polymer complexes and drug-polymer particles are useful for administering the drug(s) of the complex and particle to a subject for treatment of a disease or condition that is treatable by administering the drug.

In certain embodiments, the drug-polymer complex or drug-polymer particle is administered subcutaneously.

Representative Drug-Polymer Complexes, Drug-Polymer Particles, Drug-Polymer Formulations, and Related Methods

The following provides a description of representative drug-polymer complexes, representative particles, their formulations, methods for making the complexes, particles, and formulations, as well as methods of their use.

Poloxamer excipients containing hydrophobic and hydrophilic domains have been commonly used as surfactants. Poloxamers are triblock copolymers composed of a hydrophobic (polyoxypropylene) core (block) flanked by hydrophilic (polyoxyethylene) arms (blocks). Due to their gelling properties at physiologic temperature, poloxamers are used in various topical gel applications. The inventors have discovered that the intermolecular interactions between poloxamer and drug molecules, regardless of the hydrophobicity or hydrophilicity characteristics of the drug molecule, provide a basis for formation of stable drug-polymer particles. While the exact mechanisms of stable association of hydrophobic and hydrophilic drug to the hydrophobic core and hydrophilic arms of poloxamers remains unknown, without being bound to theory, it is likely that the inter- and intra-molecular interactions of poloxamers (e.g., F127) are mediated by van der Waals hydrophobic interactions, hydrogen bonding, and with the bound water molecules being displaced through a melt-quench process. As described herein, the melt-quench process, enables the addition of hydrophobic drugs, such as lopinavir (LPV; Log D 4.6) and ritonavir (RTV; Log D 5.7), as well as hydrophilic drugs, such as lamivudine (3TC; Log D −1.1), to substitute the inter-poloxamer (i.e., poloxamer-poloxamer) interactions with poloxamer-hydrophobic drug (e.g., LPV and RTV) in the hydrophobic copolymer domains and polyxamer-hydrophilic drug (e.g., 3TC) in the hydrophilic copolymer domains of the same poloxamer molecule. By heating and melting these molecules together at high temperature (e.g., fusion) the inter-poloxamer bonds, such as van der Waals and hydrogen bonds, are displaced and residual bound water that formed hydrogen-bonding are substituted with respective drug molecules. By this method, the multi-drug combination and poloxamer complexes are formed with significant increases in stability upon cooling of the molten state complex. By size-reduction of the complex using a number of mechanical and other processes, nano-sized drug complexes with sustained release properties are obtained. Sustained release properties of both hydrophobic and hydrophilic drugs demonstrate that their interactions with poloxamer (drug association) are non-covalent but stable physical bonding of these drugs to poloxamer. When hydrated to provide a suspension, these particles allow for sustained release characteristics that provide long-acting behavior and colloidal sizes that are suitable for development of injectable drug combination formulations.

The melt-quench method and copolymer particle products are described and exemplified herein using one hydrophilic and two hydrophobic antiviral (HIV) drugs retained stably within this drug-polymer suspension. This approach can be used to deliver combination anti-retrovirals with varying physiochemical properties and produce a sustained release of drugs over time.

Combination therapy is of particular interest in HIV/AIDS treatment due to the emergence of drug resistance, which may also be addressed with the sustained release characteristics of this product. Anti-retrovirals span a diverse range of physiochemical properties and the combination of these drugs in a single, injectable, sustained release formulation can address treatment failure in HIV/AIDS due to resistance. In addition, by virtue of their sustained release properties, the drug copolymer particles of the invention may also improve patient compliance by removing the need for daily oral dosing.

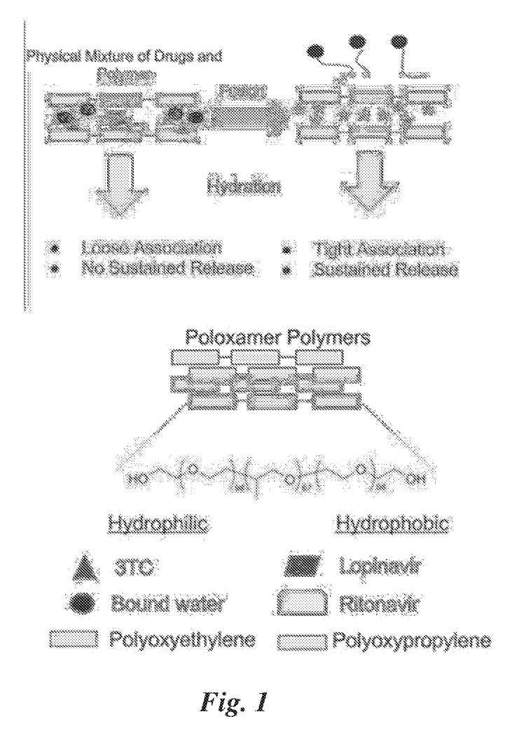

A graphical representation of a triblock copolymer having polyoxyethylene-polyoxypropylene-polyoxyethylene blocks (e.g., poloxamer) and a physical mixture of the copolymer with multiple drugs (i.e., a hydrophilic drug lamivudine and hydrophobic drugs lopinavir and ritonavir) and water is shown in FIG. 1. The hydrophilic drug lamivudine and water associate with the hydrophilic portion of the triblock copolymer (polyoxyethylene) and the hydrophobic drugs lopinavir and ritonavir associate with the hydrophobic portion of the triblock copolymer (polyoxypropylene). Fusion of triblock copolymer and hydrophobic and hydrophilic drugs provides a representative multi-drug triblock copolymer particle having strong non-bonding interactions that when hydrated result in colloidal particle formation, improved suspension characteristics, and sustained drug release from the particle. Hydration of the physical mixture reflects only a loose association between the drug and copolymer, and no sustained drug release.

Based on the following experiments, poloxamers (e.g., F127), which have polyoxyethylene and polyoxypropylene domains, are effective carriers for the delivery of hydrophilic and hydrophobic drugs. This delivery system relies on the formation of strong, nonbonding interactions between drug and polymer in the suspended state that can disrupt the intermolecular interactions of the poloxamer alone. The drug-copolymer complexes allow for increased ability to suspend hydrophobic drugs, to sustain release of hydrophobic and hydrophilic drugs, and provide advantageously sized particles. These properties make these delivery systems amenable to pharmaceutical product development.

Concentration Dependent Micellar and Gelling Behavior of Poloxamer F127 at Varying Temperatures.

Poloxamer F127 (F127) has both temperature- and concentration-dependent micelle formation (CMC) and gel phase transition (GMC). These behaviors represent the inter- and intra-molecular interactions of polymer chains in solution. Drug binding to poloxamer perturb these interactions and affect gelling temperature.

To characterize the gelling behavior of F127, multiple solutions were made at the following concentrations.

TABLE 1
Gelling behavior of F127 alone at room
temperature under varying w/v ratios.

| % (w/v) | Gel formation |
|---|---|
| 0.0001 | − |
| 0.001 | − |
| 0.01 | − |
| 0.1 | − |
| 1 | − |
| 10 | + |
| 20 | + |
| 30 | + |

Visual inspection of various concentrations of F127 in deionized water show that F127 begins to transition into the gel phase as it approaches 20% w/v or greater at room temperature. At 30% w/v, gel phase does not reverse even when stored at 4° C. Dynamic light scattering at various concentrations (room temperature) showed the presence of micelles in solutions with greater than 0.1% F127. The size of these micelles could be altered via brief sonication.

High-Resolution Microscopic Analysis of Melt-Quenched and Physically Mixed Lopinavir with Poloxamer F127.

Hydrophobic protease inhibitors (lopinavir) bind to the hydrophobic core of F127 in the molten state and suspend into colloidal particles visible with high resolution microscopy.

Hydrophobic lopinavir (with a more amenable Tₘ relative to ritonavir) underwent a melt-quench process in F127 and showed promising results of association. A 10:90 lopinavir to F127 physical mixture was ramped to 125° C. and rapidly quenched. The resulting quenched material (100 mg) was dissolved in DI water and subjected to high-resolution microscopic analysis. A control solution of lopinavir-F127 at the same concentrations were also made without fusion and dissolved in water.

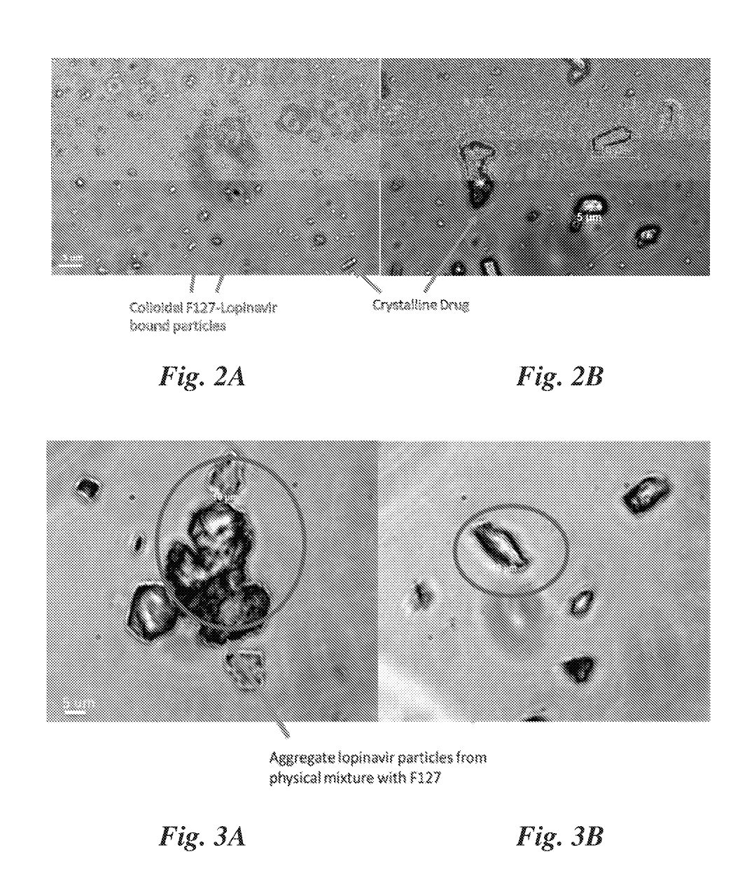

FIGS. 2A and 2B are high resolution microscopic images of an aqueous suspension containing melt-quenched F127-lopinavir particles. Colloidal and micron-sized particles and crystalline drug are observed having varying morphologies.

FIGS. 3A and 3B are high resolution microscopic images of an aqueous suspension containing drug triblock copolymer particles (lopinavir and poloxamer F127 particles) formed as a physical mixture. Large aggregate particles can be observed with irregular, faceted morphology.

Relative to lopinavir alone, the addition of F127 promotes the dissolution of lopinavir by acting as a surfactant. When drug and poloxamer are allowed to associate in the molten state, a greater population of colloidal particles is produced when viewing through high-resolution microscopy. The physical mixture of lopinavir and F127 still allows for dissolution of lopinavir, but particles are much larger. Dynamic light scattering shows strong signal and populations of colloidal particles in both physically mixed and melt-quenched F127-lopinavir suggests that nanoparticles are formed regardless of either physically mixing or mixing through fusion. However, the amount of drug associated with the particles may change. Colloidal particles from melt-quenched material are unlikely to be the micellar form of F127 as these are not seen in the physical mixture at the same concentration.

Size and Suspension Characteristics of Lopinavir-F127 Associated Particles with Varying Ratios of Drug to Polymer.

Hydrophobic lopinavir does not suspend in aqueous media. Association with F127 in the molten state, as in the copolymer particles of the invention, provides for greater supersaturation of lopinavir in suspension and non-bonding interactions sustain the release of lopinavir from the particle.

The effect of thermal treatment of lopinavir with and without F127 was evaluated. Saturated solutions of lopinavir that underwent thermal treatment alone and with varying ratios of F127 were prepared and lopinavir concentration was measured using LC-MS/MS.

TABLE 2
Maximum lopinavir concentration in suspension of various
w/w ratio formulations of lopinavir and F127 following
thermal treatment. Solutions with varying ratios of drug-
to-excipient are normalized to Lopinavir content*

| | Maximum drug concentration in |
|---|---|
| Formulation | suspension (μg/ml) |
| Lopinavir alone (reported) | 7.70 |
| Lopinavir (amorphous) | 322.00 |
| Lopinavir:F127 (10:90) | 74500.00 |
| Lopinavir:F127 (25:75) | 54000.00 |
| Lopinavir:F127 (50:50) | 82600.00 |

Thermal treatment of lopinavir results in significant improvement of solubility relative to reported values. However, addition of F127 at varying ratios produces an even greater improvement in solubility.

Particles formed through thermal association of lopinavir and F127 contained both colloidal and coarse particles. In reference to the initial microscopic analysis, the coarse particles are most likely the rod-like structures seen above, while the colloidal particles are most likely the small spherical structures. The distribution of lopinavir between colloidal and coarse particles was then investigated by separating both populations through centrifugation and measuring the concentration of lopinavir in each through LC-MS/MS. Colloidal particles were sized through dynamic light scattering with the following size distributions for the different ratios of lopinavir to F127.

TABLE 3
Gaussian size distribution of the colloidal fraction
of suspended lopinavir:F127 particles.

| Supernatant | 10:90 | 25:75 | 50:50 |
|---|---|---|---|
| Size (nm) | 174.4 ± 27.7 | 170.1 ± 12.9 | 205.6 ± 35.2 |

After separation of the two populations and analysis of lopinavir concentration, lopinavir was mostly found in the coarse particles with 86%, 95%, and 98% for 10:90, 25:75, and 50:50 weight-by-weight ratios, respectively. Remaining drug was present as colloidal particles.

After forming particles of lopinavir, the degree to which drug was bound to polymer was evaluated through equilibrium dialysis. Changing the ratio of excipient-to-drug can change the degree of association. Following 3 hours of dialysis with a molecular weight cut off of 6-8 kDa, lopinavir was found to have 22%, 84%, and 38% association for 50:50, 25:75, and 10:90 formulations, respectively.

Sustained Release of Both Hydrophobic (LPV, RTV) and Hydrophilic (3TC) Drugs can be Observed from the Polymer Matrix at Physiological Temperatures.

F127 associated particles can release both hydrophilic and hydrophobic drugs from the polymer matrix and sustain that release over time.

As an extension to the previous experiments, a 3-drug combination particle of lopinavir, ritonavir, and lamivudine (4:1:3 w/w) was formulated at a total drug:polymer ratio of 24:76. This demonstrated the release characteristics of hydrophilic and hydrophobic drugs from the polymer nanoparticles under membrane dialysis conditions at 37° C.

The combination LPV/RTV/3TV/F127 nanoparticles displayed sustained release characteristics. FIGS. 4A and 4B graphically illustrate drug release over time for a representative multi-drug triblock copolymer particle of the invention: melt-quench formed lopinavir (LPV), ritonavir (RTV), lamivudine (3TC), poloxamer F127. Release of lopinavir and ritonavir from the particle is shown in FIG. 4A and release of lamivudine from the particle is shown in FIG. 4B. The results show that the release of each drug was sustained.

In the data presented above, it can be seen that hydrophilic and hydrophobic drugs release at variable rates with hydrophobic drugs releasing much slower than hydrophilic from our in vitro dialysis experiment. These data show indicate that sustained release in vivo reflects biological milieu that is also aqueous.

The Physical State of F127 Carrier Polymers Via PXRD.

The association of drugs with polymer in the molten state produces a phase transition in the constituent materials that allows for greater association in suspension.

F127 was associated with antiretroviral drugs (LPV/RTV/3TC) using melt quench and a physical admixture of the 4 components. X-ray diffraction was performed to evaluate the crystallinity of the samples. F127 alone has primary diffraction peaks at 19.2° and 23.3°. In the physical mixture, while the diffraction pattern is dominated by the strong signal of F127, diffraction peaks attributable to the crystalline API can also be observed. Association of drugs with F127 through melt quench shows diffraction attributable to F127, but the presence of the individual components is significantly masked. This indicates that the carrier polymer remains in its original orientation, but the individual drugs may no longer be crystalline. Amorphous conversion may aid the hydration process and allow for maximal polymer:drug interactions upon suspension.

Melt-Quench Particles.

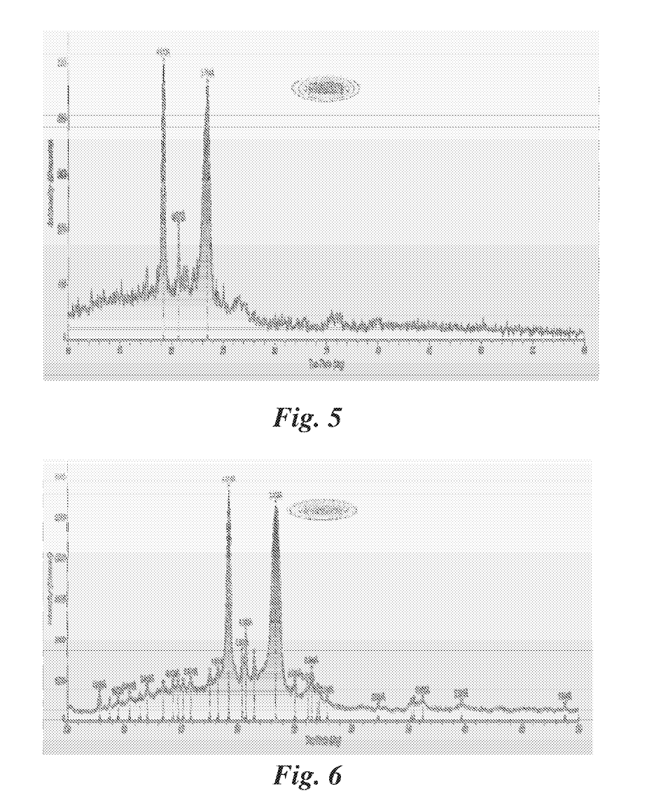

The powder X-ray diffraction pattern of a representative multi-drug triblock copolymer particle of the invention: melt-quench formed lopinavir (LPV), ritonavir (RTV), lamivudine (3TC), poloxamer F127 is shown FIG. 5. Referring to FIG. 5, absence of most diffraction peaks indicates a degree of phase transition from a crystalline to an amorphous phase of the individual drugs.

Physical Mixture.

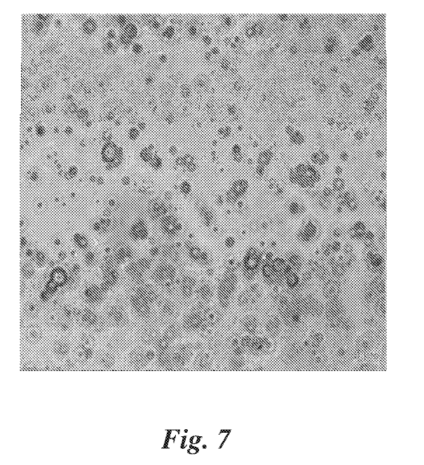

The powder X-ray diffraction pattern of a physical mixture of lopinavir (LPV), ritonavir (RTV), lamivudine (3TC), poloxamer F127 particles is shown FIG. 6. Referring to FIG. 6, individual diffraction peaks as denoted with arrows and indicate significant crystallinity in the particle (note: melt-quench particle experiments were run to 60°, but the physical mixture was run to 50° with different slit lengths (0.6, 0.6, 0.2 to 1, 1, 0.6) due to change in instrumentation).

Disruption of Intra-Polymeric Interactions of F127 Indicate New Nonbonding Interactions Between Drugs and Polymer.

Hypothesis: New nonbonding interactions between drugs and polymer disrupt the inter- and intra-polymeric interactions of F127 alone and produce a shift in the gelling temperature.

F127 has well-documented concentration- and temperature-dependent gelling upon hydration. Gelling behavior is a function of the nonbonding interactions between polymeric excipients. When these interactions are present following the addition of other molecules (such as LPV, RTV, 3TC), then the intermolecular interactions of polymer-to-polymer are preserved and drug may not be binding to polymer. In contrast, the loss of gel transition may indicate drug to polymer interactions.

Melt-quench formulations of LPV/F127, RTV/F127, 3TC/F127 and LPV/RTV/3TC/F127 were made and the gel transition temperature was observed. F127 content was fixed at 15.2% w/v and total drug content in each formulation was fixed at 24% (24:76, drug-to-polymer). 1 mL of each formulation was heated to the following temperatures and tilted to determine if a gel had formed. Gelling behavior of different drug:polymer combinations were determined to evaluate the inter- and intra-molecular interactions of polymeric chains. The results are summarized in Table 4 (positive signs (+) denote that a gel had formed at that temperature and a negative sign (−) denotes that no gel was observed).

TABLE 4
Formulation gelling behavior as a function of temperature.

| | Formulation | | | | |
|---|---|---|---|---|---|
| Temperature | LPV/RTV/ | | | | F127 |
| (° C.) | 3TC/F127 | LPV/F127 | RTV/F127 | 3TC/F127 | alone |
| 22.3 | − | − | − | − | − |
| 30 | − | − | − | + | + |
| 35 | − | − | − | + | + |
| 37.5 | − | − | + | + | + |
| 40 | − | + | + | + | + |
| 42.5 | − | + | + | + | + |
| 45 | − | + | + | + | + |
| 47.5 | − | + | + | + | − |
| 50 | − | − | + | + | − |

Addition of hydrophobic drugs (LPV and RTV) appear to have a greater impact on shifting the gel transition temperature of F127, relative to 3TC alone. This indicates that the molecular interactions between the hydrophobic cores of F127 and LPV/RTC may be strong enough to disrupt the inter-polymeric interactions of F127. 3TC alone does not appear to significantly associate with F127 as no change in the gelling temperature is observed relative to F127 alone. The formulation including all three drugs has the most significant effect on gelling, as no gelling is observed up to 50° C. F127 alone was observed to begin flowing at temperatures >47.5° C.

The effect of drug:polymer ratio on lopinavir concentration in suspension is shown in FIG. 8. Lopinavir particles with varying weight by weight ratio of F127 (10:90, 25:75, and 50:50) were prepared by the melt-quench method (fusion) followed by trituration, suspension, and sonication to provide an aqueous suspension of lopinavir/F127 particles. Lopinavir in suspension was measured in μg/mL. Panel A compares reported lopinavir solubility and measured lopinavir concentrations. Panel B compares the effect of three drug:polymer ratios on lopinavir concentration. This data shows the ability of drug:polymer suspensions to improve supersaturation and suspension characteristics of lopinavir.

The effect of drug:polymer ratio on lopinavir retention in the particle complex is shown in FIG. 9. Lopinavir particles with varying weight by weight ratio of F127 (10:90, 25:75, and 50:50) were prepared by the melt-quench method (fusion) followed by trituration, suspension, and sonication to provide an aqueous suspension of lopinavir/F127 particles that was separated by centrifugation to provide coarse (>1 micron) and colloidal (<1 micron) particles. The degree of drug retention in the complexes were evaluated after three hours of dialysis (37° C.). The results are expressed in percent of lopinavir remaining from dialysis of the supernatant (colloidal), resuspended pellet (coarse) and total complex. Greatest total association efficiency is seen with 25:75 particles.

The particle size distribution of colloidal drug:polymer complexes is shown in FIG. 10. Particle size distributions of representative multi-drug copolymer colloidal particles: melt-quench formed lopinavir (LPV), ritonavir (RTV), lamivudine (3TC), poloxamer F127 particles are compared in FIG. 10. Supernatant particle size distributions were obtained by intensity, volume, and number weighing schemes. Chi squared value for Gaussian distribution=0.471. Total drug:polymer ratio was 25:75.

Poloxamers are polymeric excipients containing hydrophobic and hydrophilic domains have been commonly used as surfactants. Due to its gelling properties at physiologic temperature, poloxamers composed of a hydrophobic core (polyoxypropylene) flanked by two hydrophilic arms (polyoxyethylene) are used in various topical gel applications. The inventors have discovered formation of a physically stable, inter molecular interactions of poloxamer and drug molecules enabled by melt-quenched control process of heating and cooling cycle—regardless of drugs' hydrophobicity or hydrophilicity characteristics, provides a basis for producing a nano-pharmaceutical composed of drug-polymer particles. These particles provide both formation of drug colloidal particles suitable for development of injectable formulation that provide long-acting behavior. This concept is demonstrated using with a hydrophilic and two hydrophobic antiviral (HIV) drugs retained stably within this drug-polymer in suspension. This approach can be used to deliver combination anti-retrovirals with varying physiochemical properties and produce a sustained release of drugs over time. Combination therapy is of particular interest in HIV/AIDS treatment due to the emergence of drug resistance, which may also be addressed with the sustained release characteristics of this product. Anti-retrovirals span a diverse range of water soluble and insoluble physiochemical properties and the combination of these drugs in single, injectable, sustained release formulation can address treatment failure in HIV/AIDS due to drug resistance.

As used herein, the term “about” refers to ±5% of the specified value.

The drug-polymer complexes, drug-polymer particles, and drug-polymer particle formulations comprise the specified components and may include other unspecified components. It will be appreciated that in other embodiments, the drug-polymer complexes, drug-polymer particles, and drug-polymer particle formulations consist of the specified components and do not include any other components. It will also be appreciated that in further embodiments, the drug-polymer complexes, drug-polymer particles, and drug-polymer particle formulations consisting essentially of the specified components and do not include other components that materially alter their properties or characteristics, such as components that would adversely affect their sustained release properties or render the compositions unsuitable for their intended purpose (e.g., therapeutic administration).

While illustrative embodiments have been illustrated and described, it will be appreciated that various changes can be made therein without departing from the spirit and scope of the invention.

1. A drug-polymer complex, comprising:
(a) one or more therapeutic agents; and
(b) a triblock copolymer having a first block that is a hydrophilic block, a second block that is a hydrophobic block, and a third block that is a hydrophilic block, wherein the second block is intermediate the first and third blocks, wherein the complex is substantially free of water.

2-4. (canceled)

5. The complex of claim 1, wherein the complex comprises one or more therapeutic agents having a Log D from about 3 to about 5, and one or more therapeutic agents having a Log D from about −2 to about 1.

6. (canceled)

7. The complex of claim 1, wherein the one or more therapeutic agents are selected from the group consisting of lopinavir, ritonavir, lamivudine, and combinations thereof.

8-9. (canceled)

10. The complex of claim 1, wherein the triblock copolymer has the formula:
H(OCH₂CH₂)ₓ(OCH₂CH(CH₃))_{y}(OCH₂CH₂)_{z}OH
wherein
x is an integer from about 10 to about 200,
y is an integer from 20 to about 80, and
z is an integer from about 10 to about 200.

11-13. (canceled)

14. The complex of claim 1, wherein the ratio of therapeutic agent to triblock copolymer is about 10:90 weight:weight.

15. The complex of claim 1, wherein the ratio of therapeutic agent to triblock copolymer is about 25:75 weight:weight.

16. The complex of claim 1, wherein the ratio of therapeutic agent to triblock copolymer is about 50:50 weight:weight.

17. A drug-polymer particle, comprising a drug-polymer complex of claim 1.

18. (canceled)

19. The particle of claim 17 having a particle size from about 1 μm to about 10 μm.

20. (canceled)

21. The particle of claim 17 having a particle size from about 50 nm to about 300 nm.

22. A pharmaceutical composition, comprising a drug-polymer particle of claim 17 and a pharmaceutically acceptable carrier.

23-24. (canceled)

25. A method for administering a therapeutic agent to a subject, comprising administering a therapeutically effective amount of a drug-polymer complex of claim 1 to a subject in need thereof.

26. (canceled)

27. A method for administering a therapeutic agent to a subject, comprising administering a therapeutically effective amount of a drug-polymer particle of claim 17 to a subject in need thereof.

28. A method for treating a disease of condition, comprising administering a therapeutically effective amount of a drug-polymer particle of claim 17 to a subject in need thereof, wherein the disease or condition is treatable by administering the therapeutic agent of the drug-polymer particle.

29. (canceled)

30. The method of claim 27, wherein the drug-polymer particle comprises a therapeutic agent selected from the group consisting of lopinavir, ritonavir, lamivudine, and combinations thereof.

31. (canceled)

32. The method of claim 27, wherein
the drug-polymer complex is administered subcutaneously.

33. A method for making a drug-copolymer complex, comprising:
(a) heating one or more therapeutic agents and a triblock copolymer having a first block that is a hydrophilic block, a second block that is a hydrophobic block, and a third block that is a hydrophilic block, wherein the second block is intermediate the first and third blocks to above the melting temperature of the therapeutic agent and triblock copolymer to provide a molten material; and
(b) cooling the molten material to provide a solid drug-polymer complex.

34. The method of claim 33, wherein heating the one or more therapeutic agents and the triblock copolymer comprises heating to 125° C. at a rate of 5° C./min.

35. (canceled)

36. The method of claim 33, wherein the solid drug-polymer complex is a drug-polymer complex of claim 1.

37-45. (canceled)

46. The method of claim 33 further comprising triturating the solid drug-polymer complex in an aqueous medium to provide a suspension of drug-polymer particles in the aqueous medium.
